(19) United States
(12) Patent Application Publication
(10) Pub. No.: US 2022/0378353 A1
(43) Pub. Date: Dec. 1, 2022

(54) INFORMATION PROCESSING APPARATUS, MEASUREMENT SYSTEM, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Tomohide HIRAGAMI, Kanagawa (JP); Kenji NAGAMIYA, Kanagawa (JP); Nobuya KITAMURA, Kanagawa (JP); Yasuyuki HOSONO, Kanagawa (JP)

(21) Appl. No.: 17/662,231

(22) Filed: May 6, 2022

(30) Foreign Application Priority Data
May 25, 2021 (JP) 2021-087935
Jan. 12, 2022 (JP) 2022-003197

(51) Int. Cl. A61B 5/26; A61B 5/00; A61B 5/282; A61B 5/308

(52) U.S. Cl. CPC A61B 5/26; A61B 5/0006; A61B 5/282; A61B 5/308

(57) ABSTRACT

An information processing apparatus comprising: at least one processor configured to: specify a contact unit in contact with a part of a body of a subject among a plurality of contact units; connect the specified contact unit to a measurement unit that measures biological information of the subject in contact with the contact unit; and acquire the biological information of the subject measured by the measurement unit connected to the contact unit.

### CROSS-REFERENCE TO RELATED APPLICATIONS

The present application claims priority under 35 U.S.C. §119 to Japanese Patent Application No. 2021-087935, filed on May 25, 2021, and Japanese Patent Application No. 2022-003197, filed on Jan. 12, 2022. The above applications are hereby expressly incorporated by reference, in their entireties, into the present application.

### BACKGROUND

### Technical Field

The present disclosure relates to an information processing apparatus, a measurement system, an information processing method, and an information processing program.

### Related Art

In the related art, a technology for measuring biological information of a subject in contact with a contact unit has been known. For example, JP2011-182973A discloses detecting brainwaves of a user wearing a hat type portable unit by a brainwave detection device included in the hat type portable unit. In addition, for example, WO2019/163028A discloses taking an electrocardiogram of a user sitting in a chair by providing an electrode for electrocardiogram measurement in each of an armrest portion and a footrest portion of the chair.

In recent years, a technology for measuring biological information of an unspecified number of subjects by a biological information measurement apparatus having functions of an electrocardiograph, a body thermometer, a blood pressure monitor, and the like arranged in the street has been desired. Particularly, for example, a technology for measuring biological information of a subject in contact with a sensor without causing the subject to be aware of the measurement by providing the sensor in a strap of a train, an armrest of a seat in Shinkansen, an airplane, and the like, and a table for a plurality of persons in a restaurant, a library, and the like has been desired.

In this case, a system configuration including a plurality of contact units (for example, straps) each comprising the sensor and a measurement unit that analyzes a biological signal acquired by the sensor and measures the analyzed biological signal as the biological information is considered. For size reduction and cost reduction of the system, a configuration in which one measurement unit can handle the plurality of contact units is preferred instead of a configuration in which the measurement unit is provided for each contact unit. For example, a technology for enabling a contact unit connected to one measurement unit to be switched and connecting the measurement unit to only a contact unit in contact with the subject among a plurality of contact units has been desired.

SUMMARY

The present disclosure provides an information processing apparatus, a measurement system, an information processing method, and an information processing program that can appropriately acquire biological information even in a case where a plurality of contact units used for measuring the biological information are present.

A first aspect according to the present disclosure is an information processing apparatus comprising at least one processor configured to specify a contact unit in contact with a part of a body of a subject among a plurality of contact units, connect the specified contact unit to a measurement unit that measures biological information of the subject in contact with the contact unit, and acquire the biological information of the subject measured by the measurement unit connected to the contact unit.

In the first aspect, in a case where a plurality of the subjects are in contact with different contact units by the part of the body, the processor may specify the contact units in contact with the plurality of subjects among the plurality of contact units for each subject.

In the first aspect, the processor may be configured to specify two or more contact units in contact with each of a plurality of parts of the body of the subject among the plurality of contact units, connect the specified two or more contact units to the measurement unit, and acquire the biological information of the subject measured by the measurement unit connected to the two or more contact units.

In the first aspect, the processor may be configured to check a conduction state for each combination of two or more contact units with which one subject comes into contact at the same time among the plurality of contact units, and specify each of the plurality of parts of the body of the subject as being in contact with the two or more contact units included in the combination that is checked to be conductive.

In the first aspect, the processor may be configured to specify the contact unit in contact with the part of the body of the subject by analyzing an image obtained by imaging the plurality of contact units by a camera.

In the first aspect, the processor may be configured to transmit the acquired biological information of the subject to a terminal apparatus possessed by the subject in contact with the specified contact unit.

In the first aspect, the processor may be configured to transmit the biological information to the terminal apparatus using wireless communication.

In the first aspect, the processor may be configured to transmit the biological information to the terminal apparatus through the part of the body of the subject in contact with the specified contact unit.

In the first aspect, the biological information may be an electrocardiogram.

In the first aspect, the subject may be a quadrupedal animal, and the processor may be configured to specify a contact unit in contact with a foot bottom portion of the subject among five or more contact units arranged on a plane.

A second aspect according to the present disclosure is a measurement system comprising the information processing apparatus according to the first aspect, a plurality of contact units with which a part of a body of a subject comes into contact, and a measurement unit that measures biological information of the subject in contact with the contact unit.

A third aspect according to the present disclosure is an information processing method executed by a computer, the method comprising specifying a contact unit in contact with a part of a body of a subject among a plurality of contact units, connecting the specified contact unit to a measurement unit that measures biological information of the subject in contact with the contact unit, and acquiring the biological information of the subject measured by the measurement unit connected to the contact unit.

A fourth aspect according to the present disclosure is an information processing program causing a computer to execute a process comprising specifying a contact unit in contact with a part of a body of a subject among a plurality of contact units, connecting the specified contact unit to a measurement unit that measures biological information of the subject in contact with the contact unit, and acquiring the biological information of the subject measured by the measurement unit connected to the contact unit.

According to the above aspects, the information processing apparatus, the measurement system, the information processing method, and the information processing program according to the aspects of the present disclosure can appropriately acquire the biological information even in a case where the plurality of contact units used for measuring the biological information are present.

### BRIEF DESCRIPTION OF THE DRAWINGS

FIG. 1 is a schematic configuration diagram of a measurement system.

FIG. 2 is a block diagram illustrating an example of a hardware configuration of an information processing apparatus.

FIG. 3 is a block diagram illustrating an example of a functional configuration of the information processing apparatus.

FIG. 4 is a specific example of the measurement system.

FIG. 5 is a diagram for describing operation of a connecting portion.

FIG. 6 is a specific example of the measurement system.

FIG. 7 is a flowchart illustrating an example of information processing.

FIG. 8 is a specific example of the measurement system that can handle a plurality of users.

FIG. 9 is a diagram for describing the operation of the connecting portion.

FIG. 10 is a specific example of the measurement system that can handle the plurality of users.

FIG. 11 is a specific example of the measurement system of which a subject is an animal.

### DETAILED DESCRIPTION

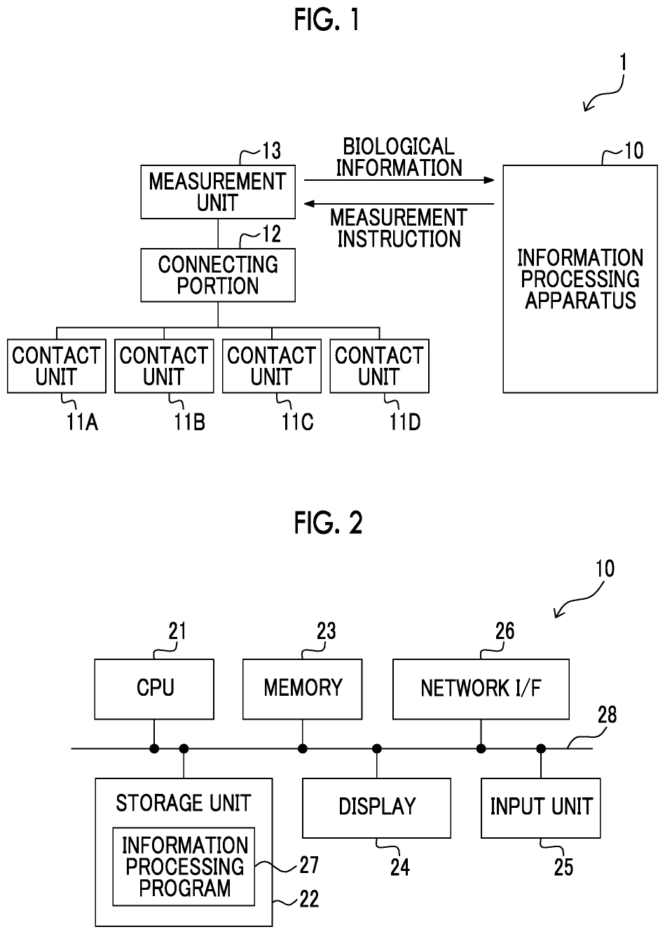

Hereinafter, an embodiment of the disclosed technology will be described in detail with reference to the drawings. First, an example of a configuration of a measurement system 1 according to the present embodiment will be described with reference to FIG. 1. As illustrated in FIG. 1, the measurement system 1 comprises an information processing apparatus 10, a plurality of contact units 11A to 11D, at least one connecting portion 12, and at least one measurement unit 13. Each of the plurality of contact units 11A to 11D is connected to the measurement unit 13 through the connecting portion 12. The number of contact units is not limited to four as illustrated in FIG. 1 and may be plural. Hereinafter, the plurality of contact units 11A to 11D will be simply referred to as “contact units 11” unless otherwise distinguished.

Each contact unit 11 is, for example, a part with which a part of a body of a subject such as a hand, an elbow, and a foot comes into contact, and comprises a sensor that detects a biological signal of the subject in contact. The measurement unit 13 has a function of measuring biological information of the subject in contact with the contact unit 11. Specifically, the measurement unit 13 analyzes the biological signal of the subject detected by the sensor comprised in the contact unit 11 and outputs the analyzed biological signal as the biological information. Examples of the biological information include an electrocardiogram, a body temperature, a blood pressure, and an arterial blood oxygen saturation level (SpO2). For example, in a case where the measurement system 1 measures the electrocardiogram of the subject, each of the plurality of contact units 11 comprises an electrocardiographic electrode as the sensor, and the measurement unit 13 analyzes the biological signal detected by the electrocardiographic electrode and outputs the analyzed biological signal as the electrocardiogram.

Here, for example, the plurality of contact units 11 according to the present embodiment are provided in straps of a train, armrests of seats in Shinkansen, an airplane, and the like, and tables for a plurality of persons in a restaurant, a library, and the like, and an unspecified number of subjects come into contact with the plurality of contact units 11. In the measurement system 1 according to the present embodiment, the contact unit 11 of a part of the plurality of contact units 11 is connected to the measurement unit 13 by the connecting portion 12, and the connected contact unit 11 can be switched. In such a manner, in the measurement system 1, by a configuration in which the plurality of contact units 11 share one measurement unit 13, size reduction and cost reduction of the measurement system 1 can be achieved.

The information processing apparatus 10 and the measurement unit 13 can communicate with each other by wired or wireless communication. The information processing apparatus 10 according to the present embodiment connects the contact unit 11 in contact with the subject to the measurement unit 13 among the plurality of contact units 11 and instructs the measurement unit 13 to measure the biological information of the subject in contact with the connected contact unit 11. In addition, the information processing apparatus 10 acquires the measured biological information from the measurement unit 13. Hereinafter, a detailed configuration of the information processing apparatus 10 will be described.

First, an example of a hardware configuration of the information processing apparatus 10 according to the present embodiment will be described with reference to FIG. 2. As illustrated in FIG. 2, the information processing apparatus 10 includes a central processing unit (CPU) 21, a non-volatile storage unit 22, and a memory 23 as a transitory storage region. In addition, the information processing apparatus 10 includes a display 24 such as a liquid crystal display, an input unit 25 such as a keyboard, a mouse, and a button, and a network interface (I/F) 26. The network I/F 26 performs wired or wireless communication with the measurement unit 13, a terminal apparatus 18 (details will be described later) possessed by the subject, and an external network (not illustrated). The CPU 21, the storage unit 22, the memory 23, the display 24, the input unit 25, and the network I/F 26 are connected to be capable of exchanging various information with each other through a bus 28 such as a system bus and a control bus.

The storage unit 22 is implemented by, for example, a storage medium such as a hard disk drive (HDD), a solid state drive (SSD), and a flash memory. The storage unit 22 stores an information processing program 27 in the information processing apparatus 10. The CPU 21 reads out the information processing program 27 from the storage unit 22, loads the information processing program 27 into the memory 23, and executes the loaded information processing program 27. The CPU 21 is an example of a processor according to the embodiment of the present disclosure. For example, a personal computer, a server computer, a tablet terminal, a smartphone, and a wearable terminal can be applied as the information processing apparatus 10.

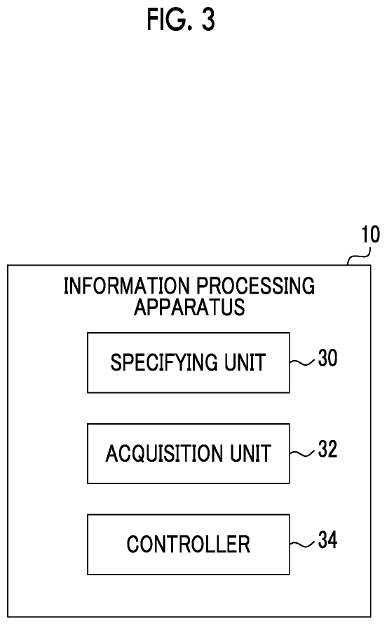

Next, an example of a functional configuration of the information processing apparatus 10 according to the present embodiment will be described with reference to FIG. 3. As illustrated in FIG. 3, the information processing apparatus 10 includes a specifying unit 30, an acquisition unit 32, and a controller 34. The CPU 21 functions as the specifying unit 30, the acquisition unit 32, and the controller 34 by executing the information processing program 27.

The specifying unit 30 specifies the contact unit in contact with the part of the body of the subject among the plurality of contact units 11. In addition, the specifying unit 30 may specify two or more contact units in contact with each of a plurality of parts of the body of the subject among the plurality of contact units 11. In this case, the specifying unit 30 may check a conduction state for each combination of two or more contact units with which one subject may come into contact at the same time among the plurality of contact units 11, and specify each of the plurality of parts of the body of the subject as being in contact with two or more contact units included in a combination that is checked to be conductive.

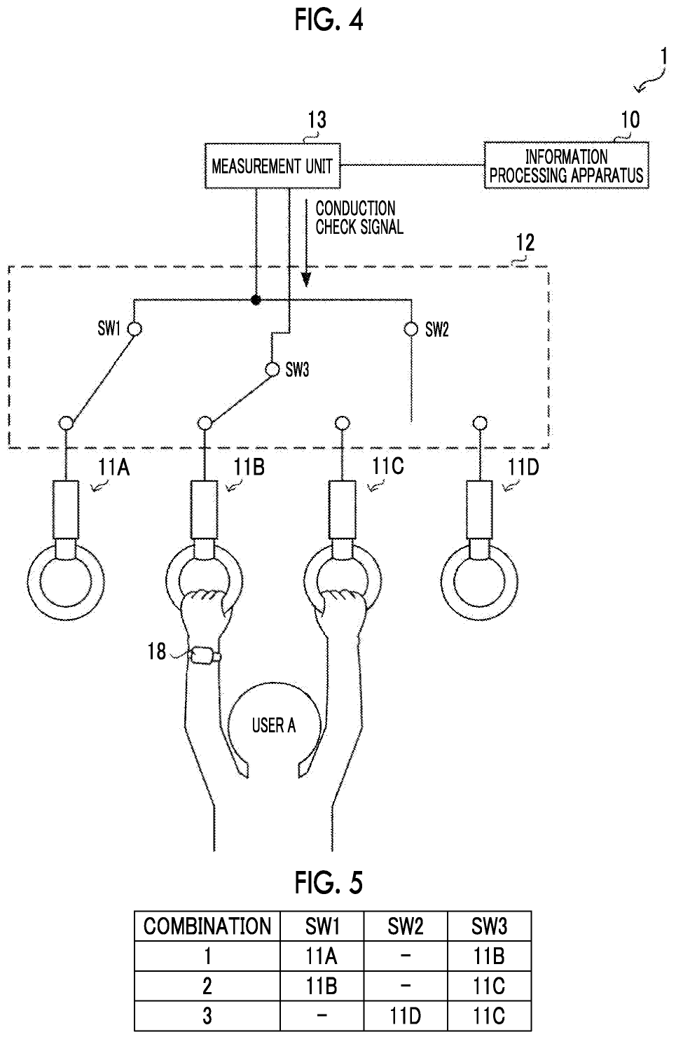

A specific example of a specifying method by the specifying unit 30 will be described with reference to FIG. 4 to FIG. 6. FIG. 4 is a configuration diagram illustrating a specific example of the measurement system 1. In the measurement system 1 in FIG. 4, in a case where a user A holds two adjacent contact units 11 (straps of a train) by both hands, and the two contact units 11 held by the user A are connected to the measurement unit 13 by the connecting portion 12, an electrocardiogram as biological information of the user A is measured. That is, two sensors comprised in the two contact units 11, respectively, in contact with the user A have roles as a positive electrode and a negative electrode, respectively, in electrocardiographic measurement. The measurement unit 13 outputs one electrocardiogram by analyzing a biological signal of the user A detected by each of the two sensors (the positive electrode and the negative electrode). The user A is an example of a subject according to the embodiment of the present disclosure.

As illustrated in FIG. 4, the connecting portion 12 includes three switches SW1 to SW3. The switch SW1 is a switch that connects the contact unit 11A or 11B to the measurement unit 13. The switch SW2 is a switch that connects the contact unit 11C or 11D to the measurement unit 13. The switch SW3 is a switch that connects the contact unit 11B or 11C to the measurement unit 13. The switches SW1 and SW2 can also be set to a state where the measurement unit 13 is not connected to any of the contact units 11.

FIG. 5 illustrates combinations of states (connection destinations of the measurement unit 13) to which each of the switches SW1 to SW3 illustrated in FIG. 4 is set at the same time. In the connecting portion 12, each of two contact units 11 (two adjacent contact units 11) with which one user A may come into contact at the same time is connected to the measurement unit 13 by switching the switches SW1 to SW3 to the state of each combination illustrated in FIG. 5. Switching of the switches SW1 to SW3 may be performed through the measurement unit 13 by instructing the measurement unit 13 by the specifying unit 30, or may be performed by directly controlling the connecting portion 12 by the specifying unit 30.

The specifying unit 30 checks the conduction state for each of Combinations 1 to 3 illustrated in FIG. 5, and specifies each of both hands of the user A as being in contact with two contact units 11 included in the combination that is checked to be conductive. Specifically, the specifying unit 30 causes the measurement unit 13 to transmit a conduction check signal (for example, a pulse signal) to any one of the two contact units 11 connected to the measurement unit 13. In a case where the user A is in contact with both of the contact unit 11 to which the conduction check signal is transmitted, and the other contact unit 11 at the same time, the conduction check signal returns to the measurement unit 13 through the body of the user A and the other contact unit 11.

Therefore, in a case where the conduction check signal has returned to the measurement unit 13, the specifying unit 30 specifies each of both hands of the user A as being in contact with the two contact units 11 connected to the measurement unit 13. On the other hand, in a case where the conduction check signal does not return to the measurement unit 13, the specifying unit 30 specifies at least one of both hands of the user A as not being in contact with at least one of the two contact units 11 connected to the measurement unit 13. The specifying unit 30 specifies the two contact units 11 in contact with both hands of the user A by sequentially checking the conduction state based on the conduction check signal for each of Combinations 1 to 3 illustrated in FIG. 5.

For example, as illustrated in FIG. 4, in a case where the user A is in contact with the contact units 11B and 11C by both hands in a state (corresponds to Combination 1 in FIG. 5) where the measurement unit 13 is connected to the contact units 11A and 11B, the conduction check signal does not return to the measurement unit 13. Therefore, the specifying unit 30 specifies at least one of both hands of the user A as not being in contact with at least one of the contact unit 11A or 11B, and checks the conduction state for the next combination (Combination 2 in FIG. 5).

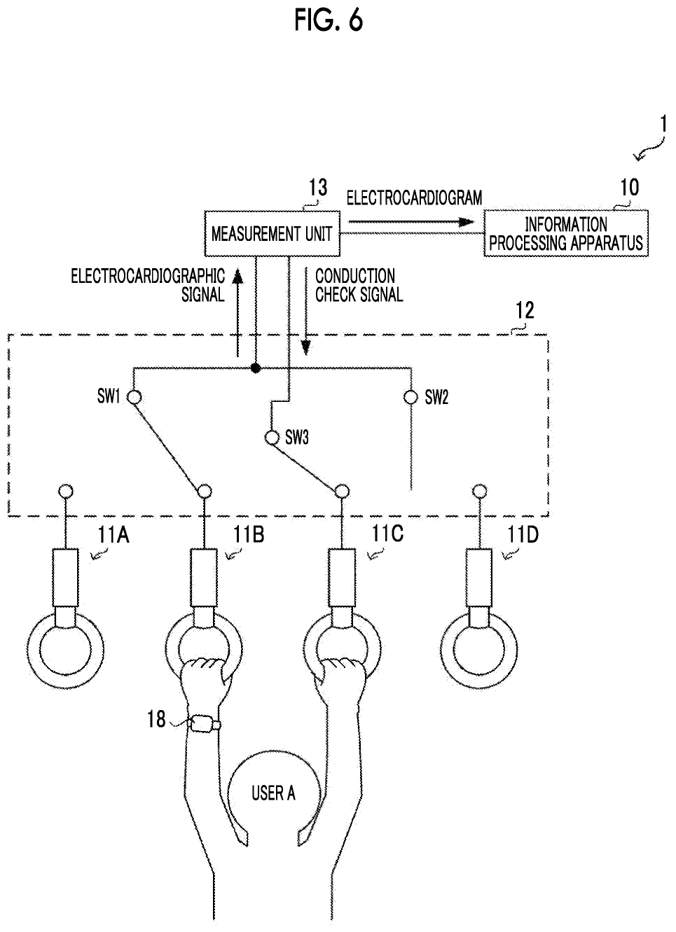

FIG. 6 is a diagram in which the switches SW1 to SW3 have switched from a state (refer to FIG. 4) corresponding to Combination 1 in FIG. 5 to a state corresponding to Combination 2 in FIG. 5. In this case, the conduction check signal transmitted to the contact unit 11C from the measurement unit 13 through SW3 returns to the measurement unit 13 through the body of the user A and the contact unit 11B. Therefore, the specifying unit 30 specifies both hands of the user A as being in contact with the contact units 11B and 11C.

The controller 34 performs a control of causing the connecting portion 12 to connect the two contact units 11 specified by the specifying unit 30 to the measurement unit 13. For example, as illustrated in FIG. 6, the controller 34 controls the connecting portion 12 to set a state where the switch SW1 is connected to the contact unit 11B, the switch SW3 is connected to the contact unit 11C, and the switch SW2 is not connected to any of the contact units 11. The control of the connecting portion 12 by the controller 34 may be performed through the measurement unit 13 or may be performed by directly controlling the connecting portion 12 by the controller 34.

The measurement unit 13 outputs an electrocardiogram by analyzing the biological signal of the subject (user A) detected by each of the two sensors comprised in the two contact units 11, respectively, connected by the connecting portion 12. The acquisition unit 32 acquires the biological information (electrocardiogram) of the subject (user A) measured by the measurement unit 13 connected to the two contact units 11.

In addition, the controller 34 may transmit the biological information of the subject (user A) acquired by the acquisition unit 32 to the terminal apparatus 18 possessed by the subject (user A) in contact with the contact units 11 specified by the specifying unit 30. Here, examples of the terminal apparatus 18 include a smartphone, a tablet terminal, and a wearable terminal.

For example, the controller 34 may transmit the biological information to the terminal apparatus 18 through the network I/F 26 using, for example, wireless communication such as Wi-Fi (registered trademark) and Bluetooth (registered trademark). In this case, in order to prevent erroneous transmission of the biological information to a terminal apparatus possessed by another subject, for example, radio waves having directivity may be transmitted toward immediately below the contact units 11 specified by the specifying unit 30.

In addition, for example, the controller 34 may transmit the biological information to the terminal apparatus 18 through the part of the body of the subject (user A) in contact with the contact units 11 specified by the specifying unit 30. For example, in a case of transmitting the biological information to a smartwatch as an example of the terminal apparatus 18, the biological information may be transmitted to the smartwatch through the hands in contact with the contact units 11 and wrists.

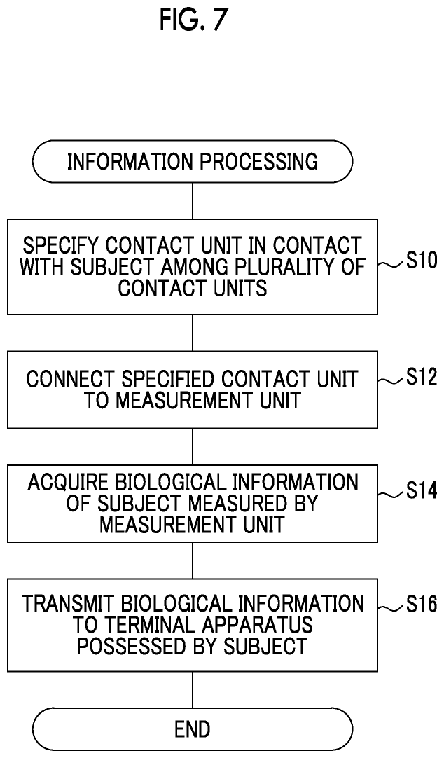

Next, actions of the information processing apparatus 10 according to the present embodiment will be described with reference to FIG. 7. In the information processing apparatus 10, information processing illustrated in FIG. 7 is executed by executing the information processing program 27 by the CPU 21. For example, the information processing is executed in a case where an execution start instruction is provided by the user through the input unit 25.

In step S10, the specifying unit 30 specifies the contact units in contact with the part of the body of the subject among the plurality of contact units 11. In step S12, the controller 34 performs the control of causing the connecting portion 12 to connect the contact units specified by the specifying unit 30 in step S10 to the measurement unit 13. In step S14, the acquisition unit 32 acquires the biological information of the subject measured by the measurement unit 13 connected to the two contact units 11 by the connecting portion 12 in step S12. In step S16, the controller 34 transmits the biological information of the subject acquired by the acquisition unit 32 in step S14 to the terminal apparatus 18 possessed by the subject in contact with the contact units specified by the specifying unit 30 in step S10, and finishes the present information processing.

As described above, the information processing apparatus 10 comprises at least one processor. The processor is configured to specify the contact unit in contact with the part of the body of the subject among the plurality of contact units 11, connect the specified contact unit to the measurement unit 13 that measures the biological information of the subject in contact with the contact unit, and acquire the biological information of the subject measured by the measurement unit 13 connected to the contact unit. That is, according to the information processing apparatus 10, even in a case where the plurality of contact units 11 used for measuring the biological information are present, it is possible to specify with which of the contact units the subject is in contact. Thus, the biological information can be appropriately acquired.

In addition, according to the information processing apparatus 10, since the plurality of contact units 11 can be configured to share one measurement unit 13, size reduction and cost reduction of the measurement system 1 can be achieved.

In the embodiment, while a form of performing the control of causing the connecting portion 12 to connect the two contact units 11 specified by the specifying unit 30 to the measurement unit 13 is described using FIG. 4 to FIG. 6 as an example, the number of contact units 11 connected to the measurement unit 13 by the connecting portion 12 is not particularly limited. For example, the connecting portion 12 may perform a control of connecting one contact unit 11 to the measurement unit 13, or may perform a control of connecting three or more contact units 11 to the measurement unit 13.

In addition, the number of contact units 11 specified by the specifying unit 30 may not necessarily match the number of contact units 11 connected to the measurement unit 13 by the connecting portion 12. For example, after the specifying unit 30 specifies two or more contact units 11 in contact with the subject at the same time based on the conduction check signal, the connecting portion 12 may perform a control of connecting the contact unit 11 of a part of the specified two or more contact units 11 to the measurement unit 13.

In addition, in the embodiment, while a form in which the specifying unit 30 specifies the contact unit 11 in contact with the subject based on the conduction check signal is described, the specifying method by the specifying unit 30 is not limited thereto. For example, the specifying unit 30 may specify the contact unit in contact with the part of the body of the subject by analyzing an image obtained by imaging the plurality of contact units 11 by a camera. In this case, even in a case where the subject is in contact with only one contact unit 11, or it is difficult to decide, in advance, the combination with which the subject may come into contact at the same time as in FIG. 5, the contact unit 11 in contact with the subject can be specified.

In addition, for example, all contact units 11 may comprise contact sensors that sense the contact of the subject, and the specifying unit 30 may specify the contact unit in contact with the part of the body of the subject by the contact sensors. In this case, in a case where a plurality of the contact units 11 specified as being in contact are present, the connecting portion 12 may perform a control of connecting one contact unit 11 to the measurement unit 13 by switching the switches to sequentially connect the contact units 11 specified as being in contact to the measurement unit 13 one by one.

In addition, in the embodiment, the biological information that is measured by the measurement unit 13 and is acquired by the acquisition unit 32 is not limited to one type and may include a plurality of types. For example, in the example in FIG. 6, a form in which a body temperature sensor is comprised in the contact unit 11B, an SpO2 sensor is comprised in the contact unit 11C, and the measurement unit 13 measures a body temperature and SpO2 based on the biological signal measured by each sensor may be applied.

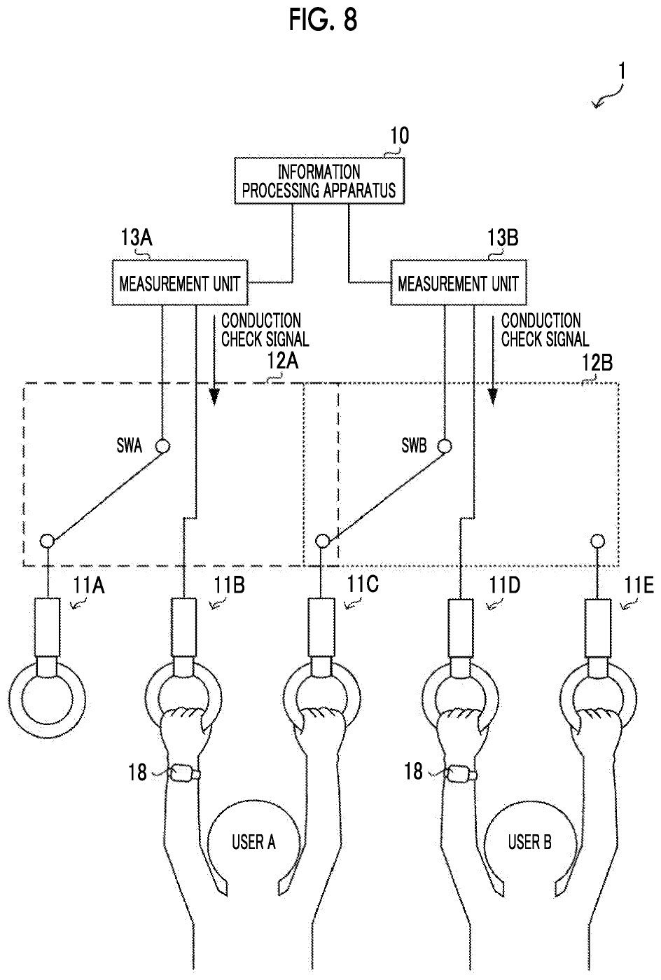

In addition, in the embodiment, while a form in which one user A is in contact with the contact units 11 by both hands in the measurement system 1 is described using FIG. 4 to FIG. 6 as an example, the present disclosure is not limited thereto. For example, the measurement system 1 can also handle a form in which each of a plurality of subjects is in contact with different contact units 11 by the part of the body. That is, the specifying unit 30 may specify the contact units in contact with the plurality of subjects among the plurality of contact units 11 for each subject. Hereinafter, a specific example of the specifying method by the specifying unit 30 in this case will be described with reference to FIG. 8 to FIG. 10.

FIG. 8 is a configuration diagram illustrating a specific example of the measurement system 1 that can handle the plurality of subjects. The measurement system 1 in FIG. 8 comprises a set including three contact units 11A to 11C, one connecting portion 12A, and one measurement unit 13A and a set including three contact units 11C to 11E, one connecting portion 12B, and one measurement unit 13B. In the measurement system 1 in FIG. 8, for each set, the specifying unit 30 specifies the contact units 11 in contact with each of users A and B, and the controller 34 performs the control of connecting the contact units 11 to the measurement unit 13. The users A and B are an example of a plurality of subjects according to the embodiment of the present disclosure.

As illustrated in FIG. 8, the connecting portion 12A includes one switch SWA, and the connecting portion 12B includes one switch SWB. The switch SWA is a switch that connects the contact unit 11A or 11C to the measurement unit 13A. The switch SWB is a switch that connects the contact unit 11C or 11E to the measurement unit 13B. As illustrated in FIG. 8, one contact unit 11C positioned between the sets can be connected to both of the measurement units 13A and 13B.

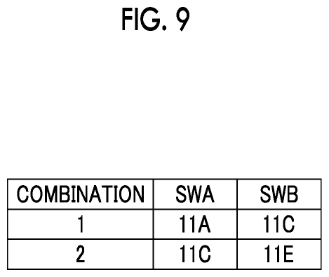

FIG. 9 illustrates combinations of states (connection destinations of the measurement units 13A and 13B) to which each of the switches SWA and SWB illustrated in FIG. 8 is set at the same time. In the connecting portions 12A and 12B, the contact units 11 in contact with the users A and B are connected to the measurement units 13A and 13B by switching the switches SWA and SWB to the state of each combination illustrated in FIG. 9.

The specifying unit 30 checks the conduction state for each of Combinations 1 and 2 illustrated in FIG. 9, and specifies a single hand of each of the users A and B as being in contact with each of two contact units 11 included in the combination that is checked to be conductive. For example, as illustrated in FIG. 8, a state where the user A is in contact with the contact units 11B and 11C by both hands, and the user B is in contact with the contact units 11D and 11E by both hands is illustrated. In this case, as illustrated in FIG. 8, in a state (corresponds to Combination 1 in FIG. 9) where the measurement unit 13A is connected to the contact units 11A and 11B, and the measurement unit 13B is connected to the contact units 11C and 11D, the conduction check signal does not return to the measurement units 13A and 13B. Therefore, the specifying unit 30 specifies both hands of the user A as being in contact with the contact units 11A and 11B and specifies both hands of the user B as not being in contact with the contact units 11C and 11D, and checks the conduction state for the next combination (Combination 2 in FIG. 9).

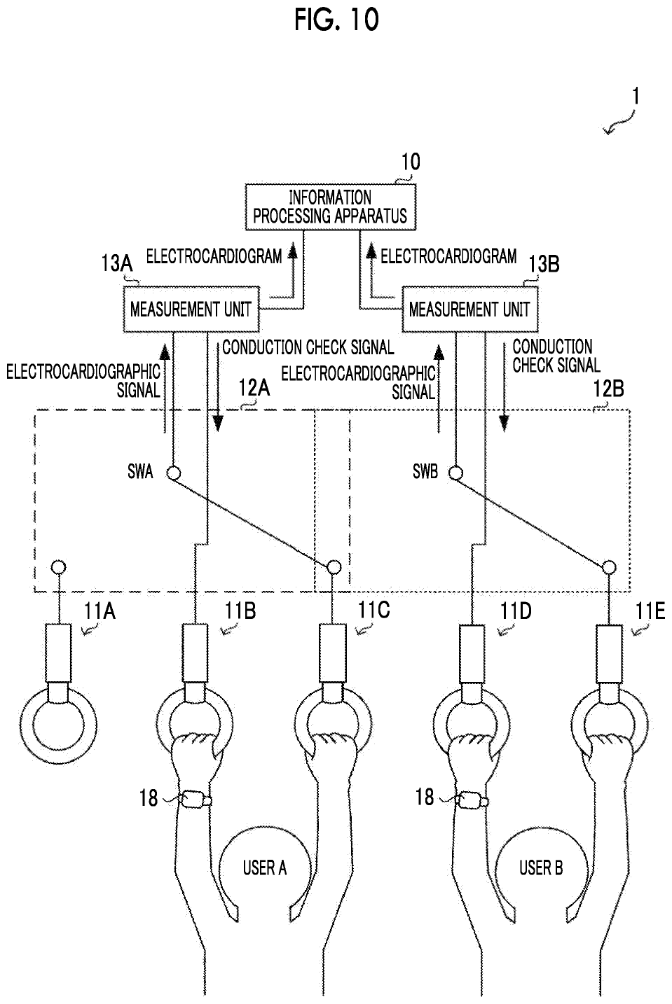

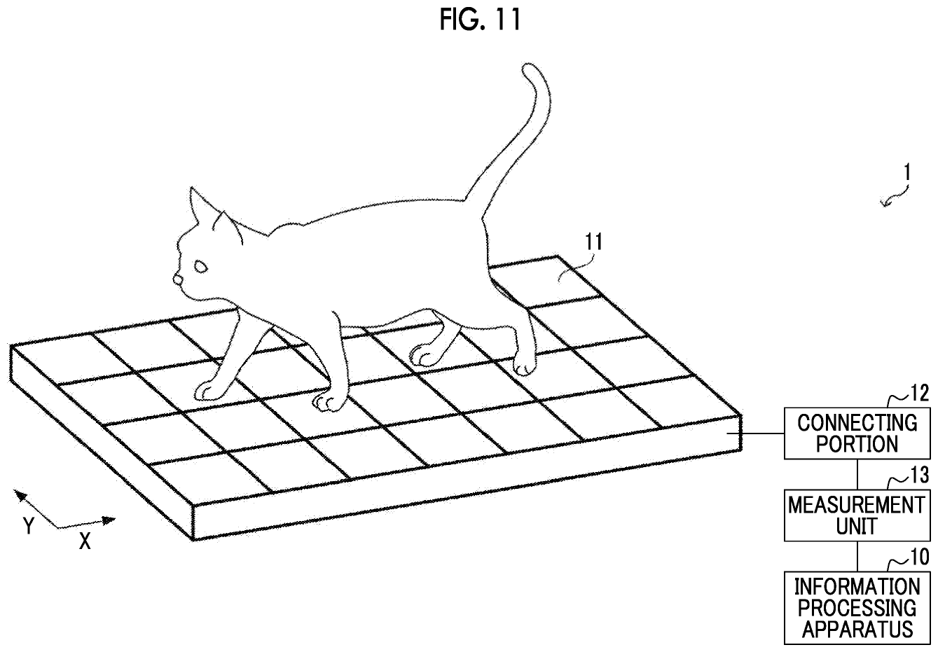

FIG. 10 is a diagram in which the switches SWA and SWB have switched from a state (refer to FIG. 8) corresponding to Combination 1 in FIG. 9 to a state corresponding to Combination 2 in FIG. 9. In this case, the conduction check signal transmitted to the contact unit 11B from the measurement unit 13A returns to the measurement unit 13A through the body of the user A, the contact unit 11C, and the switch SWA. In addition, the conduction check signal transmitted to the contact unit 11D from the measurement unit 13B returns to the measurement unit 13B through the body of the user B, the contact unit 11E, and the switch SWB. Therefore, the specifying unit 30 specifies both hands of the user A as being in contact with the contact units 11B and 11C and specifies both hands of the user B as being in contact with the contact units 11D and 11E.

The controller 34 performs a control of causing the connecting portion 12A to connect the two contact units 11B and 11C specified by the specifying unit 30 to the measurement unit 13A. In addition, the controller 34 performs a control of causing the connecting portion 12B to connect the two contact units 11D and 11E specified by the specifying unit 30 to the measurement unit 13B. Each of the measurement units 13A and 13B outputs an electrocardiogram by analyzing the biological signal of the user detected by each of the two sensors comprised in the two contact units 11, respectively, connected by each of the connecting portions 12A and 12B. The acquisition unit 32 acquires the measured electrocardiograms (biological information) of the users A and B from each of the measurement units 13A and 13B.

In addition, in the embodiment, while a form in which the subject is a person in the measurement system 1 is described, the measurement system 1 according to the embodiment of the present disclosure may be applied to an animal other than a person (hereinafter, simply referred to as the “animal”) as the subject. Hereinafter, the measurement system 1 of which the subject is the animal and that measures an electrocardiogram of the animal as the biological information will be described with reference to FIG. 11 as an example.

The measurement system 1 illustrated in FIG. 11 comprises the information processing apparatus 10, the plurality of contact units 11, the connecting portion 12, and the measurement unit 13. While forms of the plurality of contact units 11 in the measurement system 1 illustrated in FIG. 11 are different from in the measurement system 1 illustrated in FIG. 1, functions of the information processing apparatus 10, the plurality of contact units 11, the connecting portion 12, and the measurement unit 13 are the same. Thus, description will be partially omitted.

In FIG. 11, five or more contact units 11 are arranged on an XY plane as the plurality of contact units 11 and are formed in a sheet shape as a whole. Specifically, the plurality of contact units 11 are arranged in a matrix in which three or more contact units 11 are arranged in an X direction, and three or more contact units 11 are arranged in a Y direction intersecting with the X direction. Each of the plurality of contact units 11 is a part with which a foot bottom portion of the animal may come into contact, and comprises an electrocardiographic electrode that detects a biological signal of the animal in contact. The animal is standing or sitting on the plurality of contact units 11 such that four feet of the animal are in contact with any of the plurality of contact units 11.

In addition, each of the plurality of contact units 11 comprises a contact sensor (not illustrated) that senses the contact of the animal. The specifying unit 30 specifies four contact units 11 in contact with the foot bottom portions of the animal by the contact sensors comprised in the contact units 11. The controller 34 performs a control of causing the connecting portion 12 to connect the four contact units 11 specified by the specifying unit 30 to the measurement unit 13. The measurement unit 13 analyzes the biological signal of the animal detected by the electrocardiographic electrodes comprised in the four contact units 11 connected by the connecting portion 12 and outputs the analyzed biological signal as an electrocardiogram. The acquisition unit 32 acquires the electrocardiogram of the animal measured by the measurement unit 13.

In a case of the animal, a case where the animal changes a posture during the measurement of the electrocardiogram, and the contact units 11 in contact with the foot bottom portions of the animal change is assumed. Therefore, in a case where changing of the four contact units 11 in contact with the foot bottom portions of the animal is sensed by the contact sensors comprised in the contact units 11, the specifying unit 30 may specify the contact units 11 again. The controller 34 may control the connecting portion 12 to connect the four contact units 11 specified again by the specifying unit 30 to the measurement unit 13 again.

According to such a form, the four contact units 11 currently in contact with the animal among five or more contact units 11 can be specified. Thus, even in a case where the animal moves, the biological information can be appropriately acquired by following the movement. In addition, since the electrocardiogram can be measured by causing the animal to stand on the plurality of contact units 11, a burden on the animal can be reduced compared to a method of measurement by attaching the electrocardiographic electrode to a chest or the like in a state where the animal is laid down.

A quadrupedal animal can be appropriately applied as the subject of a target of the measurement system 1 illustrated in FIG. 11. Particularly, an animal having hairless parts in which the biological signal can be analyzed, in the foot bottom portions coming into contact with the contact units 11 is suitable. Examples of the animal having the hairless parts in the foot bottom portions include animals having pads in the foot bottom portion, such as Canidae, Felidae, Ursidae, Mustelidae, Procyonidae, Marsupialia, and Rodentia, and animals of which the entire foot bottom portions are hairless, such as Primates (for example, monkeys, chimpanzees, and gorillas).

In addition, in the measurement system 1 illustrated in FIG. 11, while a form in which the plurality of contact units 11 are arranged in a matrix on the XY plane is described, the present disclosure is not limited thereto. Individual shapes of the plurality of contact units 11, a shape of the plurality of contact units 11 as a whole, and an arrangement method of the plurality of contact units 11 are not particularly limited. For example, the plurality of contact units 11 may be arranged in a honeycomb structure. In addition, the plurality of contact units 11 may have different shapes. In addition, it is preferable that the number of the plurality of contact units 11 is greater than or equal to 5 and less than or equal to 500.

In addition, in the measurement system 1 illustrated in FIG. 11, while a form in which the contact units 11 and the feet of the animal are in one-to-one correspondence is illustrated, the present disclosure is not limited thereto. For example, a size (area) and a shape of each of the plurality of contact units 11 may be configured to be flexibly changed. Specifically, an animal having a foot bottom portion larger than one contact unit 11 may be handled by combining two or more contact units 11 of a part of the plurality of contact units 11 in accordance with a size and a shape of the foot bottom portion of the animal of the subject. For example, in a case where the animal having a foot bottom portion larger than one contact unit 11 is the subject, the above form may be changed to a form in which the contact units 11 and the feet of the animal are in nine-to-one correspondence, by connecting total nine contact units 11 of three arrays in the X direction and three arrays in the Y direction to one foot.

In addition, in the measurement system 1 illustrated in FIG. 11, the specifying unit 30 may specify the four contact units 11 in contact with the foot bottom portions of the animal by analyzing the image obtained by imaging the plurality of contact units 11 by the camera.

In addition, in the measurement system 1 illustrated in FIG. 11, the controller 34 may transmit the biological information of the animal acquired by the acquisition unit 32 to the terminal apparatus 18 possessed by a manager (for example, an animal keeper and a veterinarian) of the animal that is the subject. The animal that is the subject, and the terminal apparatus 18 possessed by the manager may be stored in advance in the storage unit 22 in association with each other.

In addition, in the embodiment, for example, the following various processors can be used as a hardware structure of a processing unit that executes various processing of the specifying unit 30, the acquisition unit 32, and the controller 34. The various processors include, in addition to the CPU that is a general-purpose processor functioning as various processing units by executing software (program) as described above, a programmable logic device (PLD) such as a field programmable gate array (FPGA) that is a processor having a circuit configuration changeable after manufacturing, a dedicated electric circuit such as an application specific integrated circuit (ASIC) that is a processor having a circuit configuration dedicatedly designed to execute specific processing, and the like.

One processing unit may be configured with one of the various processors or may be configured with a combination of two or more processors of the same type or different types (for example, a combination of a plurality of FPGAs or a combination of a CPU and an FPGA). In addition, a plurality of processing units may be configured with one processor.

Examples of the plurality of processing units configured with one processor include, first, as represented by a computer such as a client and a server, a form in which one processor is configured with a combination of one or more CPUs and software, and the processor functions as the plurality of processing units. Second, as represented by a system on chip (SoC) or the like, a form of using a processor that implements functions of the entire system including the plurality of processing units by one integrated circuit (IC) chip is included. In such a manner, various processing units are configured using one or more of the various processors as a hardware structure.

Furthermore, more specifically, an electric circuit (circuitry) in which circuit elements such as semiconductor elements are combined can be used as the hardware structure of the various processors.

In addition, in the embodiment, while an aspect in which the information processing program 27 is stored (installed) in advance in the storage unit 22 is described, the present disclosure is not limited thereto. The information processing program 27 may be provided in the form of a recording on a recording medium such as a compact disc read only memory (CD-ROM), a digital versatile disc read only memory (DVD-ROM), and a universal serial bus (USB) memory. In addition, the information processing program 27 may be in the form of being downloaded from an external apparatus through a network. Furthermore, in addition to the information processing program, the disclosed technology is applied to a storage medium that stores the information processing program in a non-transitory manner.

In the disclosed technology, the examples of the embodiment can also be appropriately combined. Above described contents and illustrated contents are detailed description for parts according to the embodiment of the disclosed technology and are merely an example of the disclosed technology. For example, description related to the above configurations, functions, actions, and effects is description related to an example of configurations, functions, actions, and effects of the parts according to the embodiment of the disclosed technology. Thus, unnecessary parts may be removed, new elements may be added, or parts may be replaced in the above described contents and the illustrated contents without departing from the gist of the disclosed technology.

What is claimed is:

1. An information processing apparatus comprising:
at least one processor configured to:
specify a contact unit in contact with a part of a body of a subject among a plurality of contact units;
connect the specified contact unit to a measurement unit that measures biological information of the subject in contact with the contact unit; and
acquire the biological information of the subject measured by the measurement unit connected to the contact unit.

2. The information processing apparatus according to claim 1, wherein:
in a case where a plurality of the subjects are in contact with different contact units by the part of the body,
the processor is configured to specify the contact units in contact with the plurality of subjects among the plurality of contact units for each subject.

3. The information processing apparatus according to claim 1, wherein the processor is configured to:
specify two or more contact units in contact with each of a plurality of parts of the body of the subject among the plurality of contact units;
connect the specified two or more contact units to the measurement unit; and
acquire the biological information of the subject measured by the measurement unit connected to the two or more contact units.

4. The information processing apparatus according to claim 3, wherein the processor is configured to:
check a conduction state for each combination of two or more contact units with which one subject comes into contact at the same time among the plurality of contact units; and
specify each of the plurality of parts of the body of the subject as being in contact with the two or more contact units included in the combination that is checked to be conductive.

5. The information processing apparatus according to claim 1, wherein the processor is configured to specify the contact unit in contact with the part of the body of the subject by analyzing an image obtained by imaging the plurality of contact units by a camera.

6. The information processing apparatus according to claim 1, wherein the processor is configured to transmit the acquired biological information of the subject to a terminal apparatus possessed by the subject in contact with the specified contact unit.

7. The information processing apparatus according to claim 6, wherein the processor is configured to transmit the biological information to the terminal apparatus using wireless communication.

8. The information processing apparatus according to claim 6, wherein the processor is configured to transmit the biological information to the terminal apparatus through the part of the body of the subject in contact with the specified contact unit.

9. The information processing apparatus according to claim 1, wherein the biological information is an electrocardiogram.

10. The information processing apparatus according to claim 1, wherein:
the subject is a quadrupedal animal, and
the processor is configured to specify a contact unit in contact with a foot bottom portion of the subject among five or more contact units arranged on a plane.

11. A measurement system comprising:
the information processing apparatus according to claim 1;
a plurality of contact units with which a part of a body of a subject comes into contact; and
a measurement unit that measures biological information of the subject in contact with the contact unit.

12. An information processing method executed by a computer, the method comprising:
specifying a contact unit in contact with a part of a body of a subject among a plurality of contact units;
connecting the specified contact unit to a measurement unit that measures biological information of the subject in contact with the contact unit; and
acquiring the biological information of the subject measured by the measurement unit connected to the contact unit.

13. A non-transitory computer-readable storage medium storing an information processing program causing a computer to execute a process comprising:
specifying a contact unit in contact with a part of a body of a subject among a plurality of contact units;
connecting the specified contact unit to a measurement unit that measures biological information of the subject in contact with the contact unit; and
acquiring the biological information of the subject measured by the measurement unit connected to the contact unit.
